(19) United States
(12) Patent Application Publication
(10) Pub. No.: US 2018/0064702 A1
(43) Pub. Date: Mar. 8, 2018

(54) TREATMENT OF PROGRESSIVE FORMS OF MULTIPLE SCLEROSIS WITH LAQUINIMOD

(71) Applicants: Nora Tarcic, Modiin (IL); Dan Bar-Zohar, Muttenz (CH); Liat Hayardeny, Tel-Aviv (IL); Yossi Gilgun Sherki, Bat Hefer (IL); Tali Gorfine, Tel-Aviv (IL); Volker Knappertz, Potomac, MD (US); Ella Sorani, Kadima (IL)

(72) Inventors: Nora Tarcic, Modiin (IL); Dan Bar-Zohar, Muttenz (CH); Liat Hayardeny, Tel-Aviv (IL); Yossi Gilgun Sherki, Bat Hefer (IL); Tali Gorfine, Tel-Aviv (IL); Volker Knappertz, Potomac, MD (US); Ella Sorani, Kadima (IL)

(73) Assignee: Teva Pharmaceutical Industries, Ltd., Petach-Tikva (IL)

(21) Appl. No.: 15/811,139

(22) Filed: Nov. 13, 2017

Related U.S. Application Data
(63) Continuation of application No. 14/180,173, filed on Feb. 13, 2014.
(60) Provisional application No. 61/765,394, filed on Feb. 15, 2013.
(60) Provisional application No. 61/911,106, filed on Dec. 3, 2013.

(51) Int. Cl. A61K 31/4704

(52) U.S. Cl. CPC A61K 31/4704

(57) ABSTRACT

This invention provides a method for treating a human subject afflicted with a progressive form of multiple sclerosis, comprising periodically administering to the human subject an amount of laquinimod effective to treat the human subject. This invention also provides laquinimod for use in treating a human subject afflicted with a progressive form of multiple sclerosis. This invention further provides pharmaceutical compositions and packages comprising an effective amount of laquinimod for treating a progressive form of multiple sclerosis.

This application claims benefit of U.S. Provisional Application No. 61/765,394, filed Feb. 15, 2013 and U.S. Provisional Application No. 61/911,106, filed Dec. 3, 2013, the entire content of each of which is hereby incorporated by reference herein.

Throughout this application, various publications are referred to by first author and year of publication. Full citations for these publications are presented in a References section immediately before the claims. Disclosures of the documents and publications cited and those in the References section are hereby incorporated by reference in their entireties into this application in order to more fully describe the state of the art as of the date of the invention described herein.

### BACKGROUND

### Forms of Multiple Sclerosis (MS)

Various MS disease stages and/or types are described in Multiple Sclerosis Therapeutics (Duntiz, 1999). Among them, relapsing-remitting multiple sclerosis (RRMS) is the most common form at the time of initial diagnosis. After 10-20 years, or median age 39.1 years, about half of those with RRMS gradually accumulate irreversible neurologic deficits in the absence of clinical relapses or new white matter lesions by MRI. This stage is known as secondary progressive MS (SPMS). In contrast, Primary progressive MS (PPMS) patients have progressive clinical deterioration from the onset of the disease. PPMS and SPMS are thought to be dominated by axonal degeneration in the absence of overt inflammation which is most likely a result of oxidative damage and/or increased susceptibility to injury caused by the loss of the myelin sheath (Spain 2009). Finally, Progressive-relapsing MS (PRMS) is the least common of the four disease courses, occurring in approximately 5% or so of people with MS. Like those with PPMS, PRMS patients experience disease progression from the very beginning—but they experience occasional relapses (also called attacks or exacerbations) as well. Because PRMS is progressive from onset, the doctor may initially diagnose it as PPMS, subsequently changing the diagnosis to PRMS when a relapse occurs (National Multiple Sclerosis Society Website).

Major progress has been made during the past three decades in understanding disease mechanisms in the relapsing-remitting phase of MS. This knowledge has led to effective anti-inflammatory and immunomodulatory treatments that reduce the severity and frequency of new demyelinating episodes. However, once patients have entered the progressive stage of MS, therapeutic options are currently limited to symptomatic treatments and physiotherapy. The reason for this unsatisfactory situation is that the disease mechanism driving progressive MS remain unresolved, and there is currently no animal model available that accurately reproduces this stage of MS. (Lassmann et al., 2012) There are currently a number of approved disease-modifying treatments that can reduce disease severity and progression of MS, all of which are indicated for relapsing-remitting MS. There exists a significant gap for treatment of patients afflicted with progressive forms of multiple sclerosis (Humphries, 2012).

### Laquinimod

Laquinimod is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described, for example, in U.S. Pat. No. 6,077,851.

The mechanism of action of laquinimod is not fully understood. Animal studies show it causes a Th1 (T helper 1 cell, produces pro-inflammatory cytokines) to Th2 (T helper 2 cell, produces anti-inflammatory cytokines) shift with an anti-inflammatory profile (Yang, 2004; Bruck, 2011). Another study demonstrated (mainly via the NFkB pathway) that laquinimod induced suppression of genes related to antigen presentation and corresponding inflammatory pathways (Gurevich, 2010).

Laquinimod showed a favorable safety and tolerability profile in two phase III trials for treating relapsing-remitting multiple sclerosis patients (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

### SUMMARY OF THE INVENTION

Numerous therapies which have shown benefits in relapsing-remitting multiple sclerosis patients have failed to demonstrate clinical efficacy in progressive forms of multiple sclerosis (Humphries, 2012; Wolinsky et al. 2007; Rice et al. 2000; Hawker et al., 2009; La Mantia et al., 2012). The inventors have surprisingly found that laquinimod is effective in treating patients afflicted with progressive forms of multiple sclerosis.

This invention provides a method for treating a human subject afflicted with a progressive form of multiple sclerosis, comprising periodically administering to the human subject an amount of laquinimod effective to treat the human subject.

This invention also provides laquinimod for use in treating a human subject afflicted with a progressive form of multiple sclerosis.

This invention also provides laquinimod for use in the manufacture of a medicament for treating a subject afflicted a progressive form of multiple sclerosis.

This invention also provides a pharmaceutical composition comprising an effective amount of laquinimod for treating a progressive form of multiple sclerosis.

This invention also provides a pharmaceutical composition in unit dosage form, useful in treating a subject afflicted with a progressive form of multiple sclerosis, which comprises an amount of laquinimod; which amount of said laquinimod in said composition is effective, upon administration to said subject of one or more of said unit dosage forms of said composition, to treat the subject.

This invention also provides a package comprising: a) a pharmaceutical composition comprising an amount of laquinimod; and b) instruction for use of the pharmaceutical composition to treat a subject afflicted with a progressive form of multiple sclerosis.

This invention also provides a therapeutic package for dispensing to, or for use in dispensing to, a subject afflicted with a progressive form of multiple sclerosis, which comprises: a) one or more unit doses, each such unit dose comprising an amount of laquinimod thereof, wherein the amount of said laquinimod in said unit dose is effective, upon administration to said subject, to treat the subject, and b) a finished pharmaceutical container therefor, said container containing said unit dose or unit doses, said container further containing or comprising labeling directing the use of said package in the treatment of said subject.

### BRIEF DESCRIPTION OF THE DRAWINGS

FIG. 1: shows disability progression of various forms of multiple sclerosis with time.

### DETAILED DESCRIPTION OF THE INVENTION

This invention provides a method for treating a human subject afflicted with a progressive form of multiple sclerosis, comprising periodically administering to the human subject an amount of laquinimod effective to treat the human subject.

In one embodiment, the progressive form of multiple sclerosis is Primary Progressive Multiple Sclerosis (PPMS). In another embodiment, the progressive form of multiple sclerosis is Progressive Remitting Multiple Sclerosis (PRMS). In another embodiment, the progressive form of multiple sclerosis is Secondary Progressive Multiple Sclerosis (SPMS). In another embodiment, the human subject is afflicted with a progressive form of multiple sclerosis other than a relapsing form of multiple sclerosis.

In one embodiment, the subject has an Expanded Disability Status Scale (EDSS) score of 3.0-6.5 at baseline. In another embodiment, the subject has an Expanded Disability Status Scale (EDSS) score of greater than 5.5 at baseline. In yet another embodiment, the subject has a Pyramidal Functional Systems (FS) score of 2 at baseline.

In one embodiment, the progressive form of multiple sclerosis is Secondary Progressive Multiple Sclerosis (SPMS) and the subject has an Expanded Disability Status Scale (EDSS) score of greater than 5.5 at baseline. In another embodiment, the progressive form of multiple sclerosis is Primary Progressive Multiple Sclerosis (PPMS) and the subject has an Expanded Disability Status Scale (EDSS) score of 3.0-6.5 at baseline.

In an embodiment, the amount of laquinimod is effective to inhibit progression of a symptom of the progressive form of multiple sclerosis in the subject. In another embodiment, the amount of laquinimod is effective to reduce a symptom of the progressive form of multiple sclerosis in the subject.

In one embodiment, the symptom is brain atrophy. In another embodiment, brain atrophy is measured by the change in brain volume from baseline.

In one embodiment, the symptom is impaired cognitive function. In another embodiment, cognitive function is measured by the subject's Brief International Cognitive Assessment for MS (BICAMS) score.

In one embodiment, the symptom is the subject's disability. In another embodiment, the subject's disability is measured by the Expanded Disability Status Scale (EDSS) score.

In one embodiment, laquinimod is administered via oral administration. In another embodiment, laquinimod is administered daily. In another embodiment, laquinimod is administered more often than once daily. In yet another embodiment, laquinimod is administered less often than once daily.

In an embodiment of the present invention, the amount laquinimod administered is 0.5-6.0 mg/day. In another embodiment, the amount laquinimod administered is 0.1-2.5 mg/day. In another embodiment, the amount laquinimod administered is 0.25-2.0 mg/day. In another embodiment, the amount laquinimod administered is 0.3-0.9 mg/day.

In another embodiment, the amount laquinimod administered is 0.5-1.2 mg/day. In yet another embodiment, the amount laquinimod administered is 0.6-1.8 mg/day.

In an embodiment of the present invention, the amount laquinimod administered is 0.25 mg/day. In another embodiment, the amount laquinimod administered is 0.3 mg/day. In another embodiment, the amount laquinimod administered is 0.5 mg/day. In another embodiment, the amount laquinimod administered is 0.6 mg/day. In another embodiment, the amount laquinimod administered is 0.9 mg/day. In another embodiment, the amount laquinimod administered is 1.0 mg/day. In another embodiment, the amount laquinimod administered is 1.2 mg/day. In another embodiment, the amount laquinimod administered is 1.5 mg/day. In another embodiment, the amount laquinimod administered is 1.8 mg/day. In another embodiment, the amount laquinimod administered is 2.0 mg/day. In another embodiment, the amount laquinimod administered is 2.5 mg/day. In yet another embodiment, the amount of laquinimod administered is about the amounts disclosed above.

In an embodiment of the present invention, the periodic administration continues for at least 1 week. In another embodiment, the periodic administration continues for at least 2 weeks. In another embodiment, the periodic administration continues for at least 3 weeks. In another embodiment, the periodic administration continues for at least 4 weeks. In another embodiment, the periodic administration continues for at least 5 weeks. In another embodiment, the periodic administration continues for at least 6 weeks. In another embodiment, the periodic administration continues for at least 12 weeks. In another embodiment, the periodic administration continues for at least 24 weeks. In another embodiment, the periodic administration continues for at least 3 months. In another embodiment, the periodic administration continues for at least 6 months. In yet another embodiment, the periodic administration continues for at least 15 months.

In an embodiment, laquinimod is laquinimod sodium. In another embodiment, the subject is a naive human patient to laquinimod. In another embodiment, the subject is a naive human patient to a multiple sclerosis therapy. In another embodiment, the subject is a naive human patient to any multiple sclerosis therapy.

This invention also provides laquinimod for use in treating a human subject afflicted with a progressive form of multiple sclerosis.

This invention also provides laquinimod for use in the manufacture of a medicament for treating a subject afflicted a progressive form of multiple sclerosis.

This invention also provides a pharmaceutical composition comprising an effective amount of laquinimod for treating a progressive form of multiple sclerosis.

This invention also provides a pharmaceutical composition in unit dosage form, useful in treating a subject afflicted with a progressive form of multiple sclerosis, which comprises an amount of laquinimod; which amount of said laquinimod in said composition is effective, upon administration to said subject of one or more of said unit dosage forms of said composition, to treat the subject.

This invention also provides a package comprising: a) a pharmaceutical composition comprising an amount of laquinimod; and b) instruction for use of the pharmaceutical composition to treat a subject afflicted with a progressive form of multiple sclerosis.

This invention also provides a therapeutic package for dispensing to, or for use in dispensing to, a subject afflicted with a progressive form of multiple sclerosis, which comprises: a) one or more unit doses, each such unit dose comprising an amount of laquinimod thereof, wherein the amount of said laquinimod in said unit dose is effective, upon administration to said subject, to treat the subject, and b) a finished pharmaceutical container therefor, said container containing said unit dose or unit doses, said container further containing or comprising labeling directing the use of said package in the treatment of said subject.

For the foregoing embodiments, each embodiment disclosed herein is contemplated as being applicable to each of the other disclosed embodiments. In addition, the elements recited in the pharmaceutical composition and package embodiments can be used in the method embodiments described herein and vice versa.

### Laquinimod

Laquinimod mixtures, compositions, and the process for the manufacture thereof are described in, e.g., U.S. Pat. No. 6,077,851, U.S. Pat. No. 7,884,208, U.S. Pat. No. 7,989,473, U.S. Pat. No. 8,178,127, U.S. Application Publication No. 2010-0055072, U.S. Application Publication No. 2012-0010238, and U.S. Application Publication No. 2012-0010239, each of which is hereby incorporated by reference in their entireties into this application.

Use of laquinimod for treating various conditions, and the corresponding dosages and regimens, are described in U.S. Pat. No. 6,077,851 (multiple sclerosis, insulin-dependent diabetes mellitus, systemic lupus erythematosus, rheumatoid arthritis, inflammatory bowel disease, psoriasis, inflammatory respiratory disorder, atherosclerosis, stroke, and Alzhemier's disease), U.S. Application Publication No. 2011-0027219 (Crohn's disease), U.S. Application Publication No. 2010-0322900 (Relapsing-remitting multiple sclerosis), U.S. Application Publication No. 2011-0034508 (brain-derived neurotrophic factor (BDNF)-related diseases), U.S. Application Publication No. 2011-0218179 (active lupus nephritis), U.S. Application Publication No. 2011-0218203 (rheumatoid arthritis), U.S. Application Publication No. 2011-0217295 (active lupus arthritis), and U.S. Application Publication No. 2012-0142730 (reducing fatigue, improving quality of life, and providing neuroprotection in MS patients), each of which is hereby incorporated by reference in their entireties into this application.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit can be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents (disintegrants), coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators (disintegrants) include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248. These references in their entireties are hereby incorporated by reference into this application.

General techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds).; Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is the use of laquinimod for treating a progressive form of multiple sclerosis in a human subject.

### Terms

As used herein, and unless stated otherwise, each of the following terms shall have the definition set forth below.

As used herein, “laquinimod” means laquinimod acid or a pharmaceutically acceptable salt thereof.

As used herein, an “amount” or “dose” of laquinimod as measured in milligrams refers to the milligrams of laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion.

As used herein, “about” in the context of a numerical value or range means ±10% of the numerical value or range recited or claimed.

As used herein, “effective” when referring to an amount of laquinimod refers to the quantity of laquinimod that is sufficient to yield a desired therapeutic response without undue adverse side effects (such as toxicity, irritation, or allergic response) commensurate with a reasonable benefit/risk ratio when used in the manner of this invention.

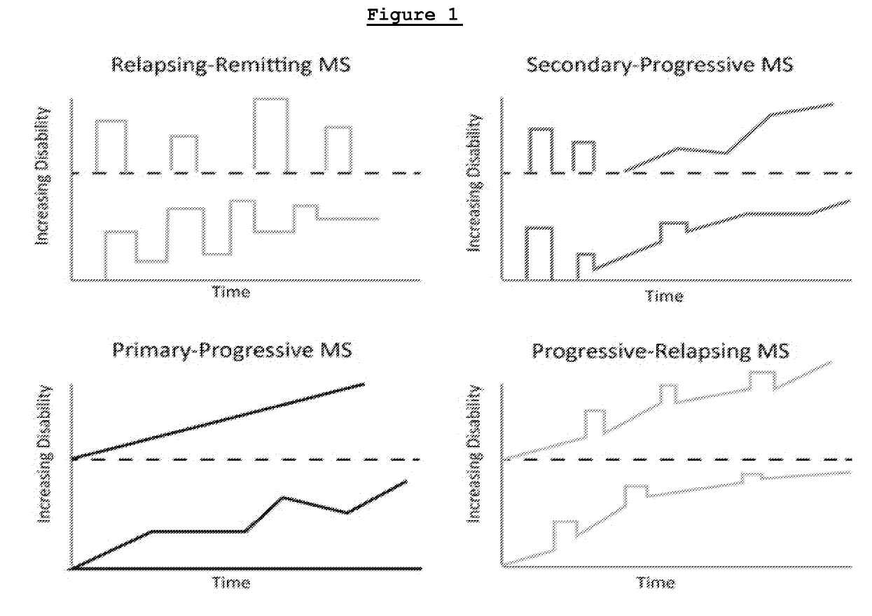

As used herein, a “relapsing form of multiple sclerosis” means a form of multiple sclerosis characterized by relapses. Of the four types of multiple sclerosis patients identified in FIG. 1, patients afflicted with Relapsing-Remitting MS (RRMS), Progressive Relapsing MS (PRMS), and Secondary Progressive MS (SPMS) can experience relapses. A “relapsing form of multiple sclerosis” or “relapsing multiple sclerosis” excludes Primary Progressive MS (PPMS) which is characterized by slowly worsening neurologic function from the beginning, with no distinct relapses or remissions (periods during which no disease progression occurs).

As used herein, “a subject afflicted with” a disease, disorder or condition means a subject who has been clinically diagnosed to have the disease, disorder or condition. For example, “a subject afflicted with PPMS” means a subject who has been clinically diagnosed to have PPMS. PPMS can be diagnosed, e.g., as defined by the Revised McDonald Criteria (Polman 2011).

As used herein, a “progressive form of multiple sclerosis” means a form of multiple sclerosis marked by progressive characteristics, i.e., disability progression and progressive neurologic decline. In another word, progressive forms of multiple sclerosis are marked by the absence of remissions. A “progressive form of multiple sclerosis” excludes Relapsing-Remitting MS (RRMS) which is characterized by clearly defined relapses followed by remissions.

Of the four disease courses identified in MS, PRMS and SPMS have both relapsing and progressive characteristics, and thus can be both a “progressive form of multiple sclerosis” and a “relapsing form of multiple sclerosis” (see FIG. 1). Accordingly, a “progressive form of multiple sclerosis” can also be a “relapsing form of multiple sclerosis” and vice versa.

“Expanded Disability Status Scale” or “EDSS” is a rating system that is frequently used for classifying and standardizing the condition of people with multiple sclerosis. The score ranges from 0.0 representing a normal neurological exam to 10.0 representing death due to MS. The score is based upon neurological testing and examination of functional systems (FS), which are areas of the central nervous system which control bodily functions. The functional systems are: Pyramidal (ability to walk), Cerebellar (coordination), Brain stem (speech and swallowing), Sensory (touch and pain), Bowel and bladder functions, Visual, Mental, and Other (includes any other neurological findings due to MS). (Kurtzke J F, 1983)

“Administering to the subject” or “administering to the (human) subject” means the giving of, dispensing of, or application of medicines, drugs, or remedies to a subject to relieve, cure, or reduce the symptoms associated with a disease, disorder or condition, e.g., a pathological condition.

“Treating” (or treat) as used herein encompasses, e.g., inducing inhibition, regression, or stasis of a disease or disorder, or lessening, suppressing, inhibiting, reducing the severity of, eliminating or substantially eliminating, or ameliorating a symptom of the disease or disorder.

“Inhibition” of disease progression or disease complication in a subject means preventing or reducing the disease progression and/or disease complication in the subject.

A “symptom” associated with a disease or disorder includes any clinical or laboratory manifestation associated with the disease or disorder and is not limited to what the subject can feel or observe.

As used herein, a subject at “baseline” is a subject prior to initiating periodic administration of laquinimod.

As used herein, a “naïve subject” or a “naïve patient” with respect to a drug or therapy means that the subject has not previously received the drug or therapy.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Clinical Trial (Phase III)—Assessment of Oral Laquinimod in Progressive Forms of Multiple Sclerosis

Introduction

Clinical trials examining the effects of laquinimod on patients having Relapsing-Remitting Multiple Sclerosis (RRMS) have demonstrated that laquinimod consistently reduced EDSS progression of disability, reduced brain atrophy and increased non-conventional MRI metrics suggestive of preservation of tissue architecture. It has now been found that laquinimod penetrates directly into the Central Nervous System (CNS) and has effects on well-defined pathways of tissue damage, apparently not involving the peripheral immune response.

Progressive MS includes Primary Progressive Multiple sclerosis (PPMS), Secondary Progressive Multiple Sclerosis (SPMS) and Progressive Relapsing MS (PRMS). The hallmarks of progressive forms of multiple sclerosis is progression, including EDSS disability progression (clinical) and axonal loss and damage, astrocytic and microglial activation, accompanied with neuronal loss (pathological).

PPMS is characterized by gradual, ongoing accrual of disability from onset. Relapses and MRI GdE-T1 activity in PPMS are relatively low compared with that in RRMS.

SPMS is the progressive stage of multiple sclerosis experienced by ex-RRMS patients and has a more heterogeneous presentation. Conversion of RRMS to SPMS is associated with early high relapse activity, followed by steady accrual of EDSS disability between relapses. Then, relapses subside (although may occur from time to time) and EDSS disability progression continues steadily (i.e., SPMS without superimposed relapses). SPMS is normally diagnosed retroactively.

Currently, treatment options for SPMS patients include potent anti-inflammatory drugs (e.g., mitoxantrone, Tysabri®, Gilenya®), IFN's (indicated for relapsing forms of MS), as well as teriflunomide (Aubagio®).

Study Duration

3-5 Years (2-4 years recruitment duration).

Study Population

Progressive Forms of Multiple Sclerosis, including Primary Progressive Multiple sclerosis (PPMS) and Secondary Progressive Multiple Sclerosis (SPMS).

Study Design

Eligible subjects are randomized into one of the following treatment arms:

0.6 mg arm: 0.6 mg laquinimod is administered orally once daily.

0.9 mg arm: 0.9 mg laquinimod is administered orally once daily.

1.2 mg arm: 1.2 mg laquinimod is administered orally once daily.

1.8 mg arm: 1.8 mg laquinimod is administered orally once daily.

Matching placebo for laquinimod arm: matching placebo for laquinimod administered once daily.

Number of Subjects/Sites

Approximately 140-240 sites and approximately 1300-2300 subjects.

### Inclusion/Exclusion Criteria

Inclusion Criteria
1. Subjects must be 25-65 years old.
2. Subjects must have a confirmed and documented diagnosis of Primary Progressive (according to McDonald), Progressive-Relapsing or Secondary Progressive (clinical definition, without relapses in the previous year) Multiple Sclerosis disease course.
3. Subjects must be ambulatory with converted Kurtzke EDSS score of 3.0-6.5.
4. Subjects must have a Pyramidal Functional Systems (FS) score of ≧2.
5. Subjects must have a threshold Timed 25-Foot Walk (T25FW) score.

Exclusion Criteria
1. Subjects with RRMS.
2. Subjects with clinically significant or unstable medical or surgical condition that would preclude safe and complete study participation, as determined by medical history, physical examination, ECG, laboratory tests or chest X-ray.
3. Known drug hypersensitivity that would preclude administration of laquinimod, such as hypersensitivity to: mannitol, meglumine or sodium stearyl fumarate.

Outcome Measures

Primary Outcome Measure

Confirmed disability progression at 3 and 6 months.

Results

This study assesses the efficacy of various daily doses of laquinimod as compared to placebo in multiple sclerosis subjects.

Daily oral administration of 0.6 mg, 0.9 mg, 1.2 mg and 1.8 mg laquinimod reduces the accumulation of physical disability in patients afflicted with a progressive form of multiple sclerosis, as compared to patients in control group receiving placebo.

Daily oral administration of 0.6 mg, 0.9 mg, 1.2 mg and 1.8 mg laquinimod reduces the accumulation of physical disability in patients afflicted with PPMS, as compared to patients in control group receiving placebo.

Daily oral administration of 0.6 mg, 0.9 mg, 1.2 mg and 1.8 mg laquinimod reduces the accumulation of physical disability in patients afflicted with SPMS, as compared to patients in control group receiving placebo.

Daily oral administration of 0.6 mg, 0.9 mg, 1.2 mg and 1.8 mg laquinimod reduces the accumulation of physical disability in patients afflicted with PRMS, as compared to patients in control group receiving placebo.

Daily oral administration of 0.6 mg, 0.9 mg, 1.2 mg and 1.8 mg laquinimod reduces the accumulation of physical disability in patients afflicted with a progressive form of multiple sclerosis, as compared to the patient at baseline.

Daily oral administration of 0.6 mg, 0.9 mg, 1.2 mg and 1.8 mg laquinimod reduces the accumulation of physical disability in patients afflicted with PPMS, as compared to the patient at baseline.

Daily oral administration of 0.6 mg, 0.9 mg, 1.2 mg and 1.8 mg laquinimod reduces the accumulation of physical disability in patients afflicted with SPMS, as compared to the patient at baseline.

Daily oral administration of 0.6 mg, 0.9 mg, 1.2 mg and 1.8 mg laquinimod reduces the accumulation of physical disability in patients afflicted with PRMS, as compared to the patient at baseline.

### Example 2

### Clinical Trial (Phase II)—Administration Of Laquinimod In Primary Progressive Multiple Sclerosis (PPMS) Subjects

This study assesses the efficacy, safety and tolerability of daily oral dose of laquinimod (0.6 mg, 1.0 mg or 1.5 mg) as compared to placebo in PPMS subjects.

Study Duration
Screening phase: Up to 1 month
Treatment Phase: At least 15 months

Three months after study completion, patients are offered the opportunity to enter into an extension phase in which they continue treatment with laquinimod daily.

Study Population

Subjects with Primary Progressive Multiple Sclerosis (approximately 500 subjects in approximately 120 centers, with about 125 subjects per study arm).

Investigational Product Route and Dosage Form
1. 0.6 mg arm: one capsule containing 0.6 mg laquinimod and the other two containing matching placebo, to be administered orally once daily.
2. 1.0 mg arm: Two capsules containing 0.5 mg laquinimod and the other containing matching placebo, to be administered orally once daily.
3. 1.5mg arm: Three capsules containing 0.5 mg laquinimod to be administered orally once daily.
4. Placebo arm: Three capsules containing placebo (0.5/0.6 mg matching) to be administered orally once daily.

Study Design

This is a multinational, multicenter, randomized, double-blind, parallel-group, placebo-controlled study to evaluate the efficacy, safety and tolerability of daily oral administration of laquinimod (0.6 mg, 1.0 mg, or 1.5 mg) in PPMS subjects. Eligible subjects are randomized in a 1:1:1:1 ratio into one of the following treatment arms:
1. Laquinimod 0.6 mg
2. Laquinimod 1.0 mg
3. Laquinimod 1.5 mg
4. Matching placebo

Subjects that stopped treatment with the study drug before the completion of Month 12 visit are considered Early Treatment Discontinuation (ETD) subjects. ETD subjects continue follow-up according to scheduled visits (until Month 12). Subjects that do not complete follow up, for any reason, are considered Early Study Discontinuation (ESD) subjects.

Subjects have the following study visits: Screening visit (−1 Month), Baseline visit (Month 0), and Months 1, 2, 3, 6, 9, 12, and every three months until study termination.

The following assessments are performed at the specified time points:
1. Vital signs are measured at each study visit.
2. A physical examination is performed at months −1, 0, 1, 3, 6 and every 6 months thereafter, ETD (if applicable), and until study completion.
3. The following safety clinical laboratory tests are performed:
   a. Complete blood count (CBC) with differential at all scheduled visits.
   b. Serum chemistry (including electrolytes, liver enzymes, urea, creatinine, glucose, total protein, albumin, direct and total bilirubin, Creatinephosphokinase (CPK), serum conventional C-reactive protein (CRP), fibrinogen and pancreatic amylase)—at all scheduled visits. Calculated Glomerular Filtration Rate (GFR) at screening and prior to each MRI scan.
   c. Lipid profile (total cholesterol, HDL, LDL, triglycerides)—at baseline (month 0) and every 12 months until completion/ETD.
   d. Serum TSH, T3 and Free T4 at baseline (month 0), month 6 and every 12 months until completion/ETD.
   e. Urinalysis at the screening visit.
   f. Serum human choriogonadotropin beta (β-hCG) in women of child-bearing potential is performed at each scheduled study visit.
   g. Urine β-hCG test is performed in women of child-bearing potential at baseline (month 0) and at each scheduled study visit thereafter.
   h. Starting after visit Month 3 a β-hCG test is performed in women of child-bearing potential every 28 (±2) days. In case of suspected pregnancy the study drug is discontinued.
4. Additional blood for analysis of serology for Hepatitis B and C viruses at baseline visit
5. Pharmacokinetic (PK) study: Blood samples for analysis of laquinimod plasma concentrations are collected at Months 1, 2, 3, 6 and 12.
6. Immunological response to treatment with laquinimod and further investigation of the potential mechanism of action—Blood samples for evaluation of the immunological response to treatment with laquinimod are collected at months: 0, 1, 3, and 12.
7. ECG is performed at months −1 (screening), 0 (baseline, three recordings 10 min apart, before first dose), 1, 2, 3, 6, 12 and every 12 months until completion/ETD.
8. Chest X-ray is performed at month −1, (if not performed within 6 months prior to the screening visit).
9. Adverse Events (AEs) are monitored throughout the study.
10. Concomitant Medications are monitored throughout the study.
11. All subjects—conventional MRI scans with gadolinium at Months 0 (14 to 7 days before Baseline), and 12. In case of ETD, or completion, an additional MRI is performed, provided no study MRI was done within the previous 3 months.
12. In case of steroid treatment, study MRI is performed before such treatment or delayed to allow a minimum of 14 days but not more than 28 days from the completion of the steroid course.
13. All subjects—MRI including 3D T1-w acquisitions of the brain and cervical cord, to measure whole brain volume, cord atrophy, thalamic atrophy, cortical atrophy and white matter (WM) atrophy at Months 0 (Baseline), 12 or ETD (if applicable).
14. Neurological evaluations, including Expanded Disability Status Scale (EDSS), Timed 25-Foot Walk (T25FW)/9-Hole Peg test (9HPG), Ambulation Index (AI), Functional systems (FS), are performed at months −1, 0, and every 3 months thereafter, ETD visit (if applicable) and until completion visit.
15. The Brief International Cognitive Assessment for MS (BICAMS), including SDMT, is evaluated at months 0 and every 12 months until completion/ETD.
16. Low contrast visual acuity (LCVA) is assessed at Months 0, 6 and 12.
17. The general health status is assessed by the EuroQoL (EQSD) questionnaire at month 0 and every 12 months until completion/ETD.
18. Quality of life is assessed by the short-Form general health survey (SF-36) subject-reported questionnaire at Months 0 and 12.
19. Relapses occurring throughout the study are confirmed/monitored.

Relapse treatment:

The allowed treatment for a relapse is intravenous methylprednisolone 1 gr/day for up to 5 consecutive days.

Ancillary studies:
1. Cerebrospinal fluid (CSF) assessment—CSF is collected from all subjects at month 0 (baseline), and month 12.
2. Pharmacogenomic (PGx) and biomarker assessment: Blood samples for PGx analysis (DNA and RNA) are collected at Baseline (or if not possible at the next possible visit) from all subjects. The objective of this study is to collect and store DNA and RNA samples for possible association analysis of genetic polymorphisms, and/or gene expression profiles with clinical or paraclinical (MRI) treatment responses to laquinimod doses, in comparison with placebo. In addition, these data are used to assess potential safety signals that may arise during the study.
3. Magnetization Transfer (MT) (selected sites) is assessed in all subjects at Months 0 (Baseline), 12, and ETD (if applicable).
4. Optical coherence tomography (OCT) evaluation (selected sites) is performed in all subjects at Months 0, 12, and ETD (if applicable) to assess retinal nerve fiber layer thickness (RNFLT).

Inclusion/Exclusion Criteria

Inclusion Criteria:
1. Subject must have a confirmed and documented PPMS diagnosis as defined by the Revised McDonald criteria (Polman 2011).
2. Subject must have lesions consistent with PPMS in either or both brain MRI and cervical spinal cord MRI.
3. Subject must have Kurtzke EDSS score of 3-6.5, inclusive, at both screening and randomization visits.
4. Subject must have evidence of clinical disability progression (retrospectively or prospectively determined) within two years prior to randomization.
5. Subject must have Function System scale score of 2 for the pyramidal system or gait impairment due to lower extremity dysfunction.
6. Subject must be between 25 to 55 years of age, inclusive.
7. Subject must be able to sign and date a written informed consent prior to entering the study.
8. Subject must be willing and able to comply with the protocol requirements for the duration of the study.
9. Women of child-bearing potential must practice an acceptable method of birth control until 30 days after the last dose of treatment was administered [acceptable methods of birth control in this study include: surgical sterilization, intrauterine devices, barrier methods (condom or diaphragm with spermicide). Hormonal methods of birth control (e.g. oral contraceptive, contraceptive patch, long-acting injectable contraceptive) are permitted but must be accompanied by a condom or a diaphragm].

Exclusion Criteria:
1. Subjects with history of MS exacerbation/attacks, including any episodes of optic neuritis.
2. Progressive neurological disorder other than PPMS.
3. Any MRI record showing presence of cervical cord compression.
4. Other MRI findings (including lesions that are atypical for PPMS) that may explain the clinical signs and symptoms
5. Relevant history of vitamin B12 deficiency.
6. Positive Human T-lymphotropic virus Type I & II (HTLV-I/II) serology.
7. Use of experimental or investigational drugs and/or participation in drug clinical studies within 6 months prior to randomization.
8. Use of immunosuppressive agents, or cytotoxic agents, including cyclophosphamide and azatioprine within 12 months prior to Baseline.
9. Previous treatment with fingolimod (Gilenya®), dimethy fumarate (Tecfidera®), teriflunomide (Aubagio®), glatiramer acetate (Copaxone®), Interferon-β (either 1a or 1b) or intravenous immunoglobulin (IVIG) within 2 months prior to randomization.
10. Previous treatment with teriflunomide (Aubagio®) within 2 years prior to randomization, unless active washout has been performed.
11. Prior use of monoclonal antibodies ever, except for:
   Natalizumab (Tysabri®), if given more than 6 months prior to randomization AND the subject is John Cunningham (JC) virus antibody test negative at Screening.
   Previous use of Rituximab, ocrelizumab, or ofatumumab, if B cell count (CD19) is higher than 80 cells/μL.
12. Use of mitoxantrone (Novantrone) within 5 years prior to Screening. Use of mitoxantrone (Novantrone) >5 years before screening is allowed in subjects with normal ejection fraction and who did not exceed the total lifetime maximal dose.
13. Previous use of laquinimod.
14. Chronic (more than 30 consecutive days or monthly dosing, e.g., with the intent of MS disease modification) systemic (IV, IM or PO) corticosteroid treatment within 2 months prior to Baseline.
15. Previous use of cladribine or alemtuzumab (Lemtrada).
16. Previous total body irradiation or total lymphoid irradiation.
17. Previous stem cell treatment, autologous bone marrow transplantation or allogenic bone marrow transplantation.
18. Use of moderate/strong inhibitors of CYP3A4 within 2 weeks prior to randomization.
19. Use of inducers of CYP3A4 within 2 weeks prior to randomization.
20. Pregnancy or breastfeeding.
21. Serum levels 3xULN of either ALT or AST at screening.
22. Serum direct bilirubin which is 2xULN at screening.
23. Subjects with a clinically significant or unstable medical or surgical condition that would preclude safe and complete study participation, as determined by medical history, physical examinations, ECG, laboratory tests or chest X-ray. Such conditions may include:
   A major cardiovascular event (e.g. myocardial infarction, acute coronary syndrome, de-compensated congestive heart failure, pulmonary embolism, coronary revascularization) that occurred during the past 6 months prior to randomization.
   Any acute pulmonary disorder.
   A CNS disorder other than MS that may jeopardize the subject's participation in the study, including such disorders that are demonstrated on the baseline MRI.
   A gastrointestinal disorder that may affect the absorption of study medication.
   Renal disease.
   Any form of acute or chronic liver disease.
   Known human immunodeficiency virus positive status.
   A history of drug and/or alcohol abuse.
   Unstable psychiatric disorder.
   Any malignancies, excluding basal cell carcinoma, in the 5 years prior to randomization.
24. A known history of sensitivity to Gadolinium (Gd).
25. GFR 60 Ml/min at screening visit.
26. Inability to successfully undergo MRI scanning.
27. Subjects who underwent endovascular treatment for Chronic Cerebrospinal Venous Insufficiency (CCSVI) within 3 months prior to randomization.
28. Known drug hypersensitivity that would preclude administration of laquinimod, such as hypersensitivity to mannitol, meglumine or sodium stearyl fumarate.

Outcome Measures

Primary Endpoint:

Brain atrophy as defined by the percentage change in brain volume from Baseline to month 12. For subjects that performed ETD, the last MRI scan is included in the analysis if performed at least 9 months under study.

Secondary Endpoints:
1. Time to confirmed disease progression (CDP), defined as increase in EDSS of ≧1 point from Baseline EDSS, if EDSS at entry is ≦5.0 or increase of ≧0.5 point, if EDSS at entry is ≧5.5. This increase should be confirmed for at least 3 months.
2. Time to CDP as measured by three types of events for each individual:
An increase by at least 20% from baseline in the score to T25FW, maintained for 3 months, or
An increase from baseline in EDSS score (1 point in subjects with baseline score 3.0 to 5.0, 0.5 points in subjects with baseline score from 5.5 to 6.0), maintained for 3 months, or
An increase of at least 30% from baseline in the 9-HPT test, maintained for 3 months.
3. The cumulative number of new T2 lesions measured at Month 0 and Month 12 between the laquinimod doses vs. placebo.
4. Change from Baseline in the BICAM score.

Exploratory Endpoints:
1. Time to confirmed disease progression (CDP), defined as increase in EDSS confirmed for at least 6 months.
2. Gadolinium enhancing lesions, new T1-hypointense lesions and changes in T2 lesion volume.
3. Advanced MRI (thalamic atrophy, cortical and WM atrophy).
4. Quality of life measures.
5. Immunological profile.

Safety Endpoints:
1. Adverse events
2. Vital signs
3. ECG findings
4. Clinical laboratory parameters

Tolerability Endpoints:
1. Proportion of subjects (%) who prematurely discontinue from the study, reason of discontinuation and the time to ETD.
2. Proportion of subjects (%) who prematurely discontinue from the study due to AEs and the time to ETD.

Statistical Considerations

Sample Size:

125 patients per arm with 1 year data provides 84% power to detect delta of 0.3 and 50% power to detect delta of 0.2 in PBVC. SD assumption is 0.8. Combining all laquinimod arms vs. placebo will provides −70% power to detect delta of 0.2 and 95% power for delta of 0.3.

Significance Level:

All statistical tests are performed at 5% nominal significance level to further define the effects estimates of Laquinimod but not for strict statistical inferences.

Results

This study assesses the efficacy of 3 daily doses of laquinimod.

Daily oral administration of 0.6 mg, 1.0 mg and 1.5 mg laquinimod reduces brain atrophy (as defined by the percentage change in brain volume from Baseline to month 12) in patients afflicted with PPMS, as compared to patients in control group receiving placebo.

Daily oral administration of 0.6 mg, 1.0 mg and 1.5 mg laquinimod reduces brain atrophy (as defined by the percentage change in brain volume from Baseline to month 12) in patients afflicted with PPMS, as compared to the patient at baseline.

Daily oral administration of 0.6 mg, 1.0 mg and 1.5 mg laquinimod reduces the accumulation of physical disability in patients afflicted with PPMS, as compared to patients in control group receiving placebo.

Daily oral administration of 0.6 mg, 1.0 mg and 1.5 mg laquinimod reduces the accumulation of physical disability in patients afflicted with PPMS, as compared to the patient at baseline.

Daily oral administration of 0.6 mg, 1.0 mg and 1.5 mg laquinimod reduces the cumulative number of new T2 lesions in patients afflicted with PPMS, as compared to patients in control group receiving placebo.

Daily oral administration of 0.6 mg, 1.0 mg and 1.5 mg laquinimod reduces the cumulative number of new T2 lesions in patients afflicted with PPMS, as compared to the patient at baseline.

Daily oral administration of 0.6 mg, 1.0 mg and 1.5 mg laquinimod improves cognitive function in patients afflicted with PPMS, as compared to patients in control group receiving placebo.

Daily oral administration of 0.6 mg, 1.0 mg and 1.5 mg laquinimod improves cognitive function in patients afflicted with PPMS, as compared to the patient at baseline.

### REFERENCES

1. Barkhof, F. (1999) “MRI in Multiple Sclerosis: Correlation with Expanded Disability Status Scale (EDSS)”, Multiple Sclerosis. 5(4):283-286 (Abstract).
2. Bjartmar and Fox (2002) “Pathological mechanisms and disease progression of multiple sclerosis: therapeutic implication”, Drugs of Today. 38:7-29.
3. Brex et al. (2002) “A longitudinal study of abnormalities on MRI and disability from multiple sclerosis”, N Engl J Med. Jan. 17, 2002 346(3):158-64.
4. Bruck et al. (2012) “Reduced astrocytic NF-kappaB activation by laquinimod protects from cuprizone-induced demyelination.” Acta Neuropathol. 124:411-424.
5. Brunmark et al. (2002) “The new orally active immunoregulator laquinimod (ABR-215062) effectively inhibits development and relapses of experimental autoimmune encephalomyelitis”, J Neuroimmunology. 130:163-172.
6. Cohen et al. (2010) “Oral fingolimod or intramuscular interferon for relapsing multiple sclerosis”. N Eng J Med; 362:402-415.
7. Comi et al. (2007) LAQ/5062 Study Group. “The Effect of Two Doses of Laquinimod on MRI-Monitored Disease Activity in Patients with Relapsing-Remitting Multiple Sclerosis: A Multi-Center, Randomized, Double-Blind, Placebo-Controlled Study”, Presented at: 59th Annual Meeting of the American Academy of Neurology; Apr. 28-May 5, 2007; Boston, Mass.
8. Comi et al. (2008) “Effect of laquinimod on MRI-monitored disease activity in patients with relapsing-remitting multiple sclerosis: a multicentre, randomised, double-blind, placebo-controlled phase IIb study”, Lancet. 371:2085-2092.
9. Comi et al. (2009) for the LAQ/5062 Clinical Advisory Board and Study Group. Long-term open extension of oral laquinimod in patients with relapsing multiple sclerosis shows favorable safety and sustained low relapse rate and MRI activity. [Ectrims abstract P443]. Mult Scler. 15(Suppl 2):S127.
10. Comi et al. (2010) for the LAQ/5062 Clinical Advisory Board and Study Group. The effect of laquinimod on MRI-monitored disease activity in patients with relapsing-remitting multiple sclerosis: a double-blind active extension of the multicentre, randomised, double-blind, parallel-group placebo-controlled study. Mult Scler. 16:1360-1366.
11. Cutter et al. (1999) “Development of a multiple sclerosis functional composite as a clinical trial outcome measure”, Brain. 122:871-882.
12. De Stefano et al. (1999) “Evidence of early axonal damage in patients with multiple sclerosis”, Neurology. 52(Suppl 2):A378.
13. Dunitz. M. (1999) Multiple sclerosis therapeutics, Ed. Rudick and Goodkin. London: Taylor & Francis, 1999.
14. Durelli et al. and the Independent Comparison of Interferon (INCOMIN) Trial Study Group. (2002) “Every-other-day interferon beta-1b versus once-weekly interferon beta-1a for multiple sclerosis: results of a 2-year prospective randomised multicentre study (INCOMIN)”, Lancet. 359:1453-60.
15. EMEA Guideline on Clinical Investigation of Medicinal Products for the Treatment of Multiple Sclerosis (CPMP/EWP/561/98 Rev. 1, November 2006).
16. Fischer et al. (1999) “The Multiple Sclerosis Functional Composite measure (MSFC): an integrated approach to MS clinical outcome assessment” Multiple Sclerosis. 5(4):244-250.
17. Fisk et al. (1994) “Measuring the Functional Impact of Fatigue: Initial Validation of Fatigue Impact Scale”, Clin Inf Dis. 18 Suppl 1:S79-83.
18. Fisk et al. (1994) “The Impact of Fatigue on Patients with Multiple Sclerosis”, Can J Neurol Sci. 21:9-14.
19. Frohman et al. (2003) “The utility of MRI in suspected MS: report of the Therapeutics and Technology Assessment Subcommittee of the American Academy of Neurology”, Neurology. Sep. 9, 2003, 61(5):602-11.
20. Giovannoni et al. (2010) “A placebo-controlled trial of oral cladribine for relapsing multiple sclerosis”, N Eng J Med. 362:416-426.
21. Golder W. (2007) “Magnetic resonance spectroscopy in clinical oncology”, Onkologie. 27(3):304-9.
22. Grossman et al. (1994) Magnetization transfer: theory and clinical applications in neuroradiology”, RadioGraphics. 14:279-290.
23. Gurevich et al. (2010) “Laquinimod suppress antigen presentation in relapsing-remitting multiple sclerosis: in-vitro high-throughput gene expression study.” J Neuroimmunol. 221:87-94.
24. Hartung et al. (2005) “Significance of neutralizing antibodies to interferon beta during treatment of multiple sclerosis: expert opinions based on the Proceedings of an International Consensus Conference”, Eur J Neurol. 12:588-601.
25. Hauser et al. (1983) “Intensive immunosuppression in progressive multiple sclerosis”, New Engl J Med. 308:173-180.
26. Hawker et al. (2009) “Rituximab in Patients with Primary Progressive Multiple Sclerosis—Results of a Randomized Double-Blind Placebo-Controlled Multicenter Trial,” Ann Neurol, 66:460-471.
27. Hohlfeld et al. (2000) “The neuroprotective effect of inflammation: implications for the therapy of multiple sclerosis”, J Neuroimmunol. 107:161-166.
28. Humphries (2012) “Progressive multiple sclerosis: The treatment gap,” Nature, 484(S10), doi:10.1038/nature 11108.
29. Jacobs et al. (1996) “Intramuscular interferon beta-1a for disease progression in relapsing multiple sclerosis”, Ann Neurol. 39:285-294.
30. Kappos et al. (2010) “A placebo-controlled trial of oral fingolimod in relapsing multiple sclerosis”, N Eng J Med. 362:387-401.
31. Kurtzke J F. (1983) “Rating neurologic impairment in multiple sclerosis: an expanded disability status scale (EDSS)”, Neurology 33(11):1444-1452.
32. La Mantia et al. (2012) “Interferon β for secondary progressive multiple sclerosis: a systematic review,” J Neurol Nerosurg Psychiatry, doi:10.1136/jnnp-2012-303291.
33. Lassmann et al. (2012) “Progressive multiple sclerosis: pathology and pathogenesis,” Natures Reviews Neurology, 8:647-656.
34. Lublin and Reingold (1996) “Defining the clinical course of multiple sclerosis”, Neurol. 46:907-911.
35. McDonald, (2001) “Guidelines from the International Panel on the Diagnosis of Multiple Sclerosis” Ann. Neurol. 50:121-127.
36. Mehta et al. (1996) “Magnetization transfer magnetic resonance imaging: a clinical review”, Topics in Magnetic Resonance Imaging 8(4):214-30.
37. Miki et al. (1999) “Relapsing-Remitting Multiple Sclerosis: Longitudinal Analysis of MR Images—Lack of Correlation between Changes in T2 Lesion Volume and Clinical Findings”, Radiology. 213:395-399.
38. Miller et al. (2007) “MRI outcomes in a placebo-controlled trial of natalizumab in relapsing MS”, Neurology. 68:1390-1401.
39. National Multiple Sclerosis Society Website, retrieved Feb. 11, 2013, http://www.nationalmssociety.org/about-multiple-sclerosis/progressive-ms/progressive-relapsing-ms/index.aspx.
40. Neuhaus et al. (2003) “Immunomodulation in multiple sclerosis: from immunosuppression to neuroprotection”, Trends Pharmacol Sci. 24:131-138.
41. Noseworthy et al. (2000) “Multiple sclerosis”, N Engl J Med. 343:938-952.
42. Noseworthy et al. (2000) “Linomide in relapsing and secondary progressive MS. Part 1: Trial Design and clinical results”, Neurology. 54:1726-1733.
43. Panitch H, Goodin D S, Francis G, Chang P, Coyle P K, O'Connor P, Monaghan E, Li D, Weinsjenker B, for the EVIDENCE (Evidence of Interferon Dose-response: European North American Comparative Efficacy) Study Group and the University of British Columbia MS/MRI Research Group. (2002) “Randomized comparative study of interferon β-1a treatment regiments in MS”, The EVIDENCE Trial. Neurology. 59:1496-1506.
44. PCT International Application Publication No. WO 1998/30227, published July 16, 1998.
45. PCT International Application Publication No. WO 2000/05250, published Feb. 3, 2000.
46. PCT International Application Publication No. WO 2000/18794, published Apr. 6, 2000.
47. PCT International Application Publication No. WO 2001/46145, published Jun. 28, 2001.
48. PCT International Application Publication No. WO 2003/048735, published Jun. 12, 2003.
49. PCT International Application Publication No. WO 2004/103297, published Dec. 2, 2004.
50. PCT International Application Publication No. WO 2006/016036, published Nov. 2, 2006.
51. PCT International Application Publication No. WO 2006/029393, published Mar. 16, 2006.
52. PCT International Application Publication No. WO 2006/029411, published Mar. 16, 2006.
53. PCT International Application Publication No. WO 2006/040155, published Apr. 20, 2006.
54. PCT International Application Publication No. WO 2006/083608, published Aug. 10, 2006.
55. PCT International Application Publication No. WO 2006/089164, published Aug. 24, 2006.
56. PCT International Application Publication No. WO 2006/116602, published Nov. 2, 2006.
57. PCT International Application Publication No. WO 2007/047863, published Apr. 26, 2007, international filing date Oct. 18, 2006.
58. PCT International Application Publication No. WO 2007/146248, published Dec. 21, 2007.
59. PCT International Application Publication No. WO 2007/146248, published Dec. 21, 2007, international filing date Jun. 12, 2007.
60. PCT International Application Publication No. WO 2009/070298, published Jun. 4, 2009.
61. PCT International Application Publication No. WO 2011/008274, published Jan. 20, 2011.
62. PCT International Application Publication No. WO 2011/022063, published Feb. 24, 2011.
63. PCT International Application Publication No. WO 2011/107583, published Sep. 9, 2011.
64. PCT International Application Publication No. WO 2012/051106, published Apr. 19, 2012.
65. Polman et al. (2005) “Diagnostic criteria for multiple sclerosis: 2005 revisions to the McDonald Criteria”, Annals of Neurology, Volume 58 Issue 6, Pages 840-846.
66. Polman et al. (2005) “Treatment with laquinimod reduces development of active MRI lesions in relapsing MS”, Neurology. 64:987-991.
67. Polman et al. (2006) “A randomized, placebo-controlled trial of natalizumab for relapsing multiple sclerosis”, N Eng J Med. 354:899-910.
68. Polman et al. (2011) “Diagnostic criteria for multiple sclerosis: 2010 Revisions to the McDonald criteria”, Ann Neurol 2011: 69:292-302.
69. Poser et al. (1983) “New Diagnostic Criteria for Multiple Sclerosis: Guidelines for Research Protocols”, Annals of Neurology, March 1983, 13(3):227-230.
70. Preiningerova J. (2009) “Oral laquinimod therapy in relapsing multiple sclerosis”, Expert Opin Investig Drugs. 18:985-989.
71. PRISMS Study Group. Randomized double-blind placebo-controlled study of interferon β-1a in relapsing/remitting multiple sclerosis. Lancet 1998;352:1498-1506.
72. Rice et al. (2000) “Cladribine and progressive MS—Clinical and MRI outcomes of a multicenter controlled trial,” Neurology, 54(5):1145-1155.
73. Rosen Y. (2007) “The Recent advances in magnetic resonance neurospectroscopy”, Neurotherapeutics. 27(3): 330-45.
74. RTT News Article dated Apr. 12, 11, entitled “Teva Pharma, Active Biotech Post Positive Laquinimod Phase 3 ALLEGRO Results”.
75. Rudick et al. (1999) “Use of the brain parenchymal fraction to measure whole brain atrophy in relapsing-remitting MS: Multiple Sclerosis Collaborative Research Group”. Neurology. 53:1698-1704.
76. Rudick, R. (1999) “Disease-Modifying Drugs for Relapsing-Remitting Multiple Sclerosis and Future Directions for Multiple Sclerosis Therapeutics”, Neurotherpatueics. 56:1079-1084.
77. Runström et al. (2002) “Laquinimod (ABR-215062) a candidate drug for treatment of Multiple Sclerosis inhibits the development of experimental autoimmune encephalomyelitis in IFN-β knock-out mice”, (Abstract), Medicon Valley Academy, Malmoe, Sweden.
78. Sandberg-Wollheim et al. (2005) “48-week open safety study with high-dose oral laquinimod in patients”, Mult Scler. 11:S154 (Abstract).
79. SIENA and SIENAX available from the FMRIB Software Library, Oxford University. Oxford, UK;http://www.fmrib.ox.ac.uk/analysis/research/siena/siena.
80. Sorenson P S. (2006) “Neutralising antibodies to interferon-β—measurement, clinical relevance, and management”, J Neurol. 253[Suppl 6]:VI/16-VI/22.
81. Sormani et al. (2004) “Measurement error of two different techniques for brain atrophy assessment in multiple sclerosis”, Neurology. 62:1432-1434.
82. Spain et al. (2009) “Recent developments in multiple sclerosis therapeutics.” BMC Medicine. 7:74.
83. Temple R. (2006) “Hy's law: predicting serious hepatoxicity”, Pharmacoepidemiol Drug Saf. 15(4):241-3.
84. The IFNB Multiple Sclerosis Study Group. (1993) Interferon beta-1b is effective in relapsing-remitting multiple sclerosis. I. Clinical results of a multicenter, randomized, double-bind, placebo-controlled trial. Neurology; 43:655-661.
85. The IFNB Multiple Sclerosis Study Group. (1993) Interferon beta-1b is effective in relapsing-remitting multiple sclerosis. II. MRI analysis results of a multicenter, randomized, double-blind, placebo-controlled trial. Neurology; 43:662-667.
86. The National MS Society (USA), The Disease Modifying Drug Brochure, Oct. 19, 2006.
87. Thone and Gold (2011) “Laquinimod: a promising oral medication for the treatment of relapsing-remitting multiple sclerosis”, Expert Opin Drug Metab Toxicol. 2011 March; 7(3): 365-70.
88. U.S. Patent Application Publication No. 2008-0207526, published Aug. 28, 2008 (Strominger et al.).
89. U.S. Patent Application Publication No. 2010-0197712, published Aug. 5, 2010 (Carlsson et al.).
90. U.S. Patent Application Publication No. 2010-0322900, published Dec. 23, 2010 (Tarcic et al.).
91. U.S. Patent Application Publication No. 2011-0027219, published Feb. 3, 2011 (Tarcic et al.).
92. U.S. Patent Application Publication No. 2011-0034508, published Feb. 10, 2011 (Liat Hayardeny).
93. U.S. Patent Application Publication No. 2011-0206782, published Aug. 25, 11 (Zhang).
94. U.S. Patent Application Publication No. 2011-0217295, published Sep. 8, 2011 (Haviv and Tarcic).
95. U.S. Patent Application Publication No. 2011-0218179, published Sep. 8, 2011 (Haviv and Tarcic).
96. U.S. Patent Application Publication No. 2011-0218203, published September 8, 2011 (Joel Kaye et al.).
97. U.S. Patent Application Publication No. 2012-0010238, published Jan. 12, 2012 (Fristedt).
98. U.S. Patent Application Publication No. 2012-0010239, published Jan. 12, 2012 (Piryatinsky et al.).
99. U.S. Patent Application Publication No. 2012-0142730, published Jun. 7, 2012 (Tarcic et al.).
100. U.S. Pat. No. 3,849,550, issued Nov. 19, 1974 (Teitelbaum et al).
101. U.S. Pat. No. 5,800,808, issued Sep. 1, 1998 (Konfino et al).
102. U.S. Pat. No. 5,858,964, issued Jan. 12, 1999 (Aharoni et al).
103. U.S. Pat. No. 5,981,589, issued Nov. 9, 1999 (Konfino et al).
104. U.S. Pat. No. 6,048,898, issued Apr. 11, 2000 (Konfino et al).
105. U.S. Pat. No. 6,054,430, issued Apr. 25, 2000 (Konfino et al).
106. U.S. Pat. No. 6,077,851, issued Jun. 20, 2000 (Bjork et al). 107. U.S. Pat. No. 6,214,791, issued Apr. 10, 2001 (Arnon et al).
108. U.S. Pat. No. 6,342,476, issued Jan. 29, 2002 (Konfino et al).
109. U.S. Pat. No. 6,362,161, issued Mar. 26, 2002 (Konfino et al).
110. U.S. Pat. No. 7,566,767, issued Jul. 28, 2009 (Strominger et al.).
111. U.S. Pat. No. 7,589,208, issued Sep. 15, 2009 (Jansson et al).
112. U.S. Patent no. 7,884,208, issued Feb. 8, 2011 (Frenkel et al.).
113. U.S. Pat. No. 7,989,473, issued Aug. 2, 2011 (Patashnik et al.).
114. U.S. Pat. No. 8,178,127, issued May 15, 2012 (Safadi et al.).
115. U.S. Pat. No. 8,252,993, issued Aug. 28, 2012 (Gant and Shahbaz).
116. US Food and Drug Administration, Center for Drug Evaluation and Research. Peripheral and Central Nervous System (PCNS) Advisory Committee. US Department of Health and Human Services 2006. Briefing Document. Biogen Idec Biologics Marketing Application STN 125104/15. Natalizumab (Tysabri) for Multiple Sclerosis. Dated Feb. 9, 2006. Pages 45-48.
117. Wolinsky et al. (2007) “Glatiramer Acetate in Primary Progressive Multiple Sclerosis: Results of a Multinational, Multicenter, Double-Blind Placebo-Controlled Trial,” Annals of Neurology, 61(1): 14-24.
118. Yang et al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.
119. Zou et al. (2002) “Suppression of experimental autoimmune neuritis by ABR-215062 is associated with altered Th1/Th2 balance and inhibited migration of inflammatory cells into the peripheral nerve tissue”, Neuropharmacology. 42:731.

What is claimed is:

1. A method for treating a human subject afflicted with a progressive form of multiple sclerosis, comprising periodically administering to the human subject an amount of laquinimod effective to treat the human subject.

2. The method of claim 1, wherein the progressive form of multiple sclerosis is Primary Progressive Multiple Sclerosis (PPMS).

3. The method of claim 1, wherein the progressive form of multiple sclerosis is Progressive Remitting Multiple Sclerosis (PRMS).

4. The method of claim 1, wherein the progressive form of multiple sclerosis is Secondary Progressive Multiple Sclerosis (SPMS).

5. The method of claim 3 or 4, wherein the human subject is afflicted with a progressive form of multiple sclerosis other than a relapsing form of multiple sclerosis.

6. The method of any one of claims 1-5, wherein the subject has an Expanded Disability Status Scale (EDSS) score of 3.0-6.5 at baseline.

7. The method of any one of claims 1-6, wherein the subject has an Expanded Disability Status Scale (EDSS) score of greater than 5.5 at baseline.

8. The method of claim 1, wherein the progressive form of multiple sclerosis is Secondary Progressive Multiple Sclerosis (SPMS) and the subject has an Expanded Disability Status Scale (EDSS) score of greater than 5.5 at baseline.

9. The method of claim 1, wherein the progressive form of multiple sclerosis is Primary Progressive Multiple Sclerosis (PPMS) and the subject has an Expanded Disability Status Scale (EDSS) score of 3.0-6.5 at baseline.

10. The method of any one of claims 1-9, wherein the subject has a Pyramidal Functional Systems (FS) score of 2 at baseline.

11. The method of any one of claims 1-10, wherein the amount of laquinimod is effective to inhibit progression of a symptom of the progressive form of multiple sclerosis in the subject.

12. The method of any one of claims 1-10, wherein the amount of laquinimod is effective to reduce a symptom of the progressive form of multiple sclerosis in the subject.

13. The method of claims 11 and 12, wherein the symptom is brain atrophy.

14. The method of claim 13, wherein brain atrophy is measured by the change in brain volume from baseline.

15. The method of claims 11 and 12, wherein the symptom is impaired cognitive function.

16. The method of claim 15, wherein cognitive function is measured by the subject's Brief International Cognitive Assessment for MS (BICAMS) score.

17. The method of claims 11 and 12, wherein the symptom is the subject's disability.

18. The method of claim 17, wherein the subject's disability is measured by the Expanded Disability Status Scale (EDSS) score.

19. The method of any one of claims 1-18, wherein laquinimod is administered via oral administration.

20. The method of any one of claims 1-19, wherein laquinimod is administered daily.

21. The method of any one of claims 1-19, wherein laquinimod is administered more often than once daily.

22. The method of any one of claims 1-19, wherein laquinimod is administered less often than once daily.

23. The method of any one of claims 1-22, wherein the amount laquinimod administered is 0.1-2.5 mg/day.

24. The method of claim 23, wherein the amount laquinimod administered is 0.6-1.8 mg/day.

25. The method of claim 24, wherein the amount laquinimod administered is 0.6 mg/day, 0.9 mg/day, 1.0 mg/day, 1.2 mg/day, 1.5 mg/day or 1.8 mg/day.

26. The method of any one of claims 1-25, wherein the periodic administration continues for at least 3 months.

27. The method of claim 26, wherein the periodic administration continues for at least 6 months.

28. The method of claim 27, wherein the periodic administration continues for at least 15 months.

29. The method of any one of claims 1-28, wherein laquinimod is laquinimod sodium.

30. The method of any one of claims 1-29, wherein the subject is a naive human patient to laquinimod.

31. Laquinimod for use in treating a human subject afflicted with a progressive form of multiple sclerosis.

32. Laquinimod for the manufacture of a medicament for use in treating a subject afflicted a progressive form of multiple sclerosis.

33. A pharmaceutical composition comprising an effective amount of laquinimod for treating a progressive form of multiple sclerosis.

34. A pharmaceutical composition in unit dosage form, useful in treating a subject afflicted with a progressive form of multiple sclerosis, which comprises an amount of laquinimod; which amount of said laquinimod in said composition is effective, upon administration to said subject of one or more of said unit dosage forms of said composition, to treat the subject.

35. A package comprising:
a) a pharmaceutical composition comprising an amount of laquinimod; and
b) instruction for use of the pharmaceutical composition to treat a subject afflicted with a progressive form of multiple sclerosis.

36. A therapeutic package for dispensing to, or for use in dispensing to, a subject afflicted with a progressive form of multiple sclerosis, which comprises:
a) one or more unit doses, each such unit dose comprising an amount of laquinimod thereof, wherein the amount of said laquinimod in said unit dose is effective, upon administration to said subject, to treat the subject, and
b) a finished pharmaceutical container therefor, said container containing said unit dose or unit doses, said container further containing or comprising labeling directing the use of said package in the treatment of said subject.
